(19) United States
(12) Patent Application Publication
(10) Pub. No.: US 2023/0118099 A1
(43) Pub. Date: Apr. 20, 2023

(54) Personalised Medicine Treatment Program

(71) Applicant: Closed Loop Medicine Ltd., Cambridge (GB)

(72) Inventors: James Matthew Siddle, London (GB); Leo Freitas, Newcastle upon Tyne (GB); David Tonatiuh Cox, London (GB); Brett Mark Fisher, Greenwich (GB); Paul Goldsmith, London (GB)

(73) Assignee: Closed Loop Medicine Ltd., Cambridge (GB)

(21) Appl. No.: 17/961,898

(22) Filed: Oct. 7, 2022

Related U.S. Application Data
(60) Provisional application No. 63/255,711, filed on Oct. 14, 2021.

(51) Int. Cl. G16H 20/10

(52) U.S. Cl. CPC G16H 20/10

(57) ABSTRACT

A method of producing an intermediate program representation for an executable personalised medicine treatment program, comprising: providing a domain specific language environment comprising a personalised-medicine programming language syntax and a user interface; selecting a medical knowledge database for a medical condition; receiving user input to build, using the personalised-medicine programming language syntax and the medical knowledge database, a treatment package for implementing one or more treatment modalities for the medical condition, the treatment package including: treatment objectives defining terminating conditions of the one or more treatment modalities; safety guardrails for ensuring patient safety; and treatment titration definitions for modulating the one or more treatment modalities; and validating the treatment package to demonstrate that the treatment objectives, safety guardrails and treatment titration definitions will be satisfied during execution of the personalised medicine treatment program.

### FIELD

The present disclosure relates to a personalised medicine treatment program and a method of forming an intermediate program representation for a personalised medicine treatment program.

### BACKGROUND

There is a clear trend towards personalised medicine, with new treatments emerging that provide individualised treatment programmes based on an individual patient's drug response, for example.

There is also a trend towards automated medicine, with software as a medical device (SaMD), such as mobile apps, playing a greater role in patient treatment. SaMD is even starting to play a role in making treatment decisions, such as changing drug doses.

Medical technology is safety critical and heavily regulated, with medical device development being highly procedural, gated, documentation heavy, and thus, expensive and time consuming to create and maintain. In contrast, medical technology companies often require rapid and cost-effective delivery of medical device software and updates thereto.

It would be desirable to produce medical device software with inherent safety built in. It would also be desirable if the inherent safety also extended to personalised medical device software that can amend an individual patient's treatment according to their personal preferences and their individual response to the treatment. The disclosed apparatus and methods may achieve one or more of these objectives.

### SUMMARY

According to a first aspect of the present disclosure there is provided a method of producing an intermediate program representation for an executable personalised medicine treatment program, comprising:

providing a domain specific language environment comprising a personalised-medicine programming language syntax and a user interface;

selecting a medical knowledge database for a medical condition;

receiving user input to build, using the personalised-medicine programming language syntax and the medical knowledge database, a treatment package for implementing one or more treatment modalities for the medical condition, the treatment package including:

treatment objectives defining terminating conditions of the one or more treatment modalities;

safety guardrails for ensuring patient safety; and

treatment titration definitions for modulating the one or more treatment modalities; and

validating the treatment package to demonstrate that the treatment objectives, safety guardrails and treatment titration definitions will be satisfied during execution of the personalised medicine treatment program.

The method may further comprise outputting the validated treatment package as the intermediate program representation.

Validating the treatment package may comprise performing well-formedness checks against semantic criteria using one or more formal methods.

Building the treatment package may comprise defining the treatment objectives, safety guardrails and/or treatment titration definitions using formal methods.

The treatment objectives, safety guardrails and/or treatment titration definitions may comprise a logical construct for evaluation during execution of the personalised medicine treatment program. The logical construct may comprise one or more formal methods.

The logical construct may be suitable for evaluation during execution using one or more formal methods.

The method may further comprise generating the personalised medicine treatment program from the intermediate program representation.

he method may further comprise receiving user input to define one or more of: patient measurements for the one or more treatment modalities; dosages for the one or more treatment modalities; explicit treatment objectives; explicit treatment ambitions defining one or more invariant properties for satisfying throughout the treatment for the one or more treatment modalities; and explicit treatment titration definitions.

The safety guardrails may comprise explicit safety guardrails defined by one or more explicit treatment objectives and/or one or more explicit treatment ambitions.

The method may further comprise compiling the treatment package.

Compiling the treatment package may comprise: expanding implicitly defined syntactic and semantic constructs within the user defined treatment package; and/or inferring and overriding information from linkages within the treatment package.

Compiling the treatment package may comprises defining implicit safety guardrails in the treatment package.

The implicit safety guardrails may include one or more of: an implicit adverse side effect treatment objective requiring immediate termination of the treatment if an adverse side effect occurs during execution of the personalised medicine treatment program; an implicit toxicity treatment ambition requiring a modality dosage to remain within a safe toxicity range, as defined by the medical knowledge database, throughout execution of the personalised medicine treatment program; an implicit sensor treatment ambition requiring sensor measurements to remain within a plausible measurement range throughout execution of the personalised medicine treatment program; and an implicit side effect combination treatment objective requiring termination of the treatment if a combination of measured side effects indicative of an overdose is determined during execution of the personalised medicine treatment program.

Receiving user input may comprise receiving user input to define one or more personal side effect tolerances. The implicit safety guardrails may include a desirable side effect treatment ambition derived from personal side effect tolerances.

The method may further comprise receiving user input to personalise the treatment package.

Receiving user input to personalise the treatment package may comprise one or more of: receiving user input to personalise one or more of the treatment titration definitions; receiving user input to personalise one or more initial treatment conditions; and receiving user input to define one or more personal side effect tolerances.

The method may further comprise receiving user input to select or define the medical knowledge database

The medical knowledge database may be defined in the personalised medicine programming language syntax

The medical knowledge database may comprise medical data for the one or more treatment modalities for the medical condition.

The medical data may include any of: dosage data, safety data and side effect data for each treatment modality.

The treatment objectives, safety guardrails and/or treatment titration definitions may comprise a logical predicate.

The logical predicate may comprise a dependence on a program state of the personalised medicine treatment program.

The treatment titration definitions may comprise one or more treatment titration criteria for evaluation during execution of the personalised medicine treatment program.

The treatment titration criteria may comprise instructions for modulating the treatment comprising modulating any of: a measurement frequency; a dose frequency; and/or a dose amount.

The treatment titration criteria may have a dependency on: one or more side effect measurements; and/or one or more physiological measurements.

The treatment titration definitions may specify a titration beat frequency for evaluating treatment modulation definitions during execution of the personalised medicine treatment program.

The intermediate program form may be configured to indicate a compliance with one or more regulatory approved dosage ranges during execution of the personalised medicine treatment program to enable operation of the personalised medicine treatment program in one of a plurality of modes.

According to a second aspect of the present disclosure there is provided an intermediate program representation for an executable personalised medicine treatment program, comprising:

a treatment package for implementing one or more treatment modalities for a medical indication, the treatment package including:

treatment objectives defining terminating conditions of the one or more treatment modalities;

safety guardrails for ensuring patient safety; and

treatment titration definitions for modulating the one or more treatment modalities,

wherein the treatment package is built using a medical knowledge database and a personalised-medicine programming language syntax of a domain specific language environment.

The treatment objectives, safety guardrails and/or treatment titration definitions may comprise a logical construct for evaluation during execution of the personalised medicine treatment program. The logical construct may comprise one or more formal methods.

The treatment package may comprise one or more of: patient measurements for the one or more treatment modalities; dosages for the one or more treatment modalities; explicit treatment objectives; explicit treatment ambitions defining one or more invariant properties for satisfying throughout the treatment for the one or more treatment modalities; and explicit treatment titration definitions.

The treatment package may further comprise a personalisation program for personalising the treatment package.

The personalisation program may be configured to: personalise one or more of the treatment titration definitions; personalise one or more initial treatment conditions; and/or define one or more personal side effect tolerances.

The treatment package may further comprise the medical knowledge database.

The medical knowledge database may comprise medical data for the one or more treatment modalities for the medical condition.

The medical data may include any of: dosage data, safety data and side effect data for each treatment modality.

The treatment objectives, safety guardrails and/or treatment titration definitions may comprise a logical predicate.

The logical predicate may comprise a dependence on a program state of the personalised medicine treatment program.

The treatment titration definitions may comprise one or more treatment titration criteria for evaluation during execution of the personalised medicine treatment program.

The treatment titration criteria may comprise instructions for modulating the treatment comprising modulating any of: a measurement frequency; a dose frequency; and/or a dose amount.

The treatment titration criteria have a dependency on: one or more side effect measurements; and/or one or more physiological measurements.

The treatment titration definitions may specify a titration beat frequency for evaluating treatment modulation definitions during execution of the personalised medicine treatment program.

According to a third aspect of the present disclosure there is provided a personalised medicine treatment program comprising any of the intermediate program forms disclosed herein.

The personalised medicine treatment program may further comprise an execution engine configured to execute the intermediate program form.

The execution engine may be configured to evaluate a logical construct of the treatment objectives, safety guardrails and/or treatment titration definitions during execution of the personalised medicine program.

The execution engine may be configured to evaluate the logical construct using one or more formal methods.

The logical construct may comprise a dependency on a program state of the personalised medicine treatment program. The execution engine may be configured to evaluate the logical construct against the program state during execution of the personalised medicine program.

The execution engine may be configured to terminate the personalised medicine treatment program if: a logical construct of a treatment objective returns a true value; and/or a logical construct of a mandatory treatment ambition returns a false value.

The execution engine may be configured to output a warning if a desirable treatment ambition returns a false value.

The execution engine may be configured to evaluate the logical constructs according to one or more beat steps defined in the intermediate program representation.

The execution engine may be configured to operate the personalised medicine treatment program in one of a plurality of modes based on a current dosage of the one or more treatment modalities.

The current dosage may be provided by the program state.

The execution engine may be configured to operate the personalised medicine treatment program in any of: an automate mode if the current dosage is within a regulatory approved dosage range; a drive mode if the current dosage is outside the regulatory approved dosage range and within a drive dosage range; and an inform mode if the current dosage is outside both the regulatory approved dosage range and the drive dosage range.

The execution engine may be configured to determine if the current dosage is within the regulatory approved dosage range and/or the drive dosage range based on either: receiving the regulatory approved dosage range and/or the drive dosage range from the intermediate program representation and comparing with the current dosage as indicated by the program state; or evaluating a logical construct of the intermediate program representation, wherein the logical construct defines a comparison between the current dosage with regulatory approved dosage range and/or the drive dosage range.

According to a fourth aspect of the present disclosure there is provided a method of treatment comprising executing any of the personalised medicine treatment programs disclosed herein.

According to a fifth aspect of the present disclosure there is provided a domain specific language environment for producing a personalised medicine treatment program for treating a medical condition with one or more treatment modalities, comprising:

a personalised-medicine programming language syntax, a user interface and an interface with a medical knowledge database for the medical condition; and

personalised-medicine programming language semantics comprising a set of declarative intents including:

treatment objective intents defining terminating conditions of the one or more treatment modalities;

safety guardrail intents for ensuring patient safety; and treatment titration definition intents for modulating the one or more treatment modalities.

### BRIEF DESCRIPTION OF THE DRAWINGS

One or more embodiments will now be described by way of example only with reference to the accompanying drawings in which:

FIG. 1 illustrates an apparatus according to an embodiment of the present disclosure;

FIG. 2 illustrates a method of receiving user input to build a treatment package according to an embodiment of the present disclosure;

FIG. 3 illustrates a method of defining a treatment knowledge database according to an embodiment of the present disclosure;

FIG. 4 illustrates a method of defining the pathway program 30 according to an embodiment of the present disclosure;

FIG. 5 illustrates a method of defining a personalisation program according to an embodiment of the present disclosure;

FIG. 6 illustrates a compilation and validation process according to an embodiment of the present disclosure;

FIG. 7 illustrates a process of, or an algorithm for, executing a DSL personalised medicine treatment program according to an embodiment of the present disclosure; and

FIGS. 8A and 8B illustrate how the variation in amlodipine dosage and side effect levels in response to execution of treatment titration criteria by a personalised medicine treatment program according to an embodiment of the present disclosure.

### DETAILED DESCRIPTION

A domain specific language (DSL) is a bespoke language, with syntax and semantics created specifically to capture and represent concepts relevant to its field of use. A DSL can succinctly and correctly express what the domain needs, at the cost of limiting user choices. For example functional intent can declare what is needed as opposed to defining instructions on how to achieve said intent.

A Domain Specific Language environment is a software environment that provides a means for coding, validating, and compiling programs written in a domain specific programming language syntax (referred to as “the DSL syntax” herein). In this disclosure the “domain” is generally personalised medicine unless otherwise indicated.

DSL syntax is a set of rules that describe what a correct program in the domain specific language looks like. In this disclosure, the DSL syntax is generally a personalised-medicine DSL syntax unless otherwise indicated.

An execution engine is a software package that forms part of a personalised medicine treatment program. The execution engine together with an intermediate program form may form the personalised medicine treatment program. The execution engine may be considered as a core platform which executes the code of the intermediate program form.

An execution environment comprises the components that are used together with the code of the personalised medicine treatment program to make the complete system: the processors, networks, operating system, virtual machine etc.

Formal methods is a mathematical technique used to construct correct software, for example in the specification, development, and verification of software. Formal methods can be used to provide a mathematical characterisation of programming language semantics, hence grounding a program's meanings clearly, and enabling analysis, such as those achieved through the disclosed safety guardrail guarantees. As described herein, formal methods will be considered to relate to the formal methods described in Woodcock et al, “Formal methods: Practice and experience” ACM Computing Surveys, Volume 41, Issue 4, October 2009, which describes the state of the art in the industrial use of formal methods.

An Intermediate Program Form or Intermediate Program Representation is a representation of an expanded and validated software program written in the DSL. The intermediate program form can be executed by an execution engine in a suitable execution environment potentially after further transformations. The intermediate program form may be considered as a precursor to a production-software program. Example intermediate program forms include an executable file, an installation package, a script file for execution in a runtime environment and a set of low-level instructions to be run on a virtual machine e.g. Java bytecode.

A medical condition is a diagnosed condition or disease.

A medical knowledge database is a database providing information describing medical conditions that are treated by one or more treatment modalities, with associated characteristics and constraints based on a trusted source, such as the British National Formulary (BNF), the National Institute for Health & Care Excellence (NICE) guidelines, authoritative textbooks or practice body guidelines such as the American Academy of Neurology for example.

Personalised medicine is the provision of individualised treatment programmes based on an individual patient response to a treatment. The term medicine may refer to a drug therapy or a non-drug therapy such as behavioural therapy or treatment with a medical device.

A personalised medicine treatment program is the production software product that runs on a patient's device to deliver personalised treatment to the patient for a specific medical condition.

A treatment modality is one treatment option for treating a specific medical condition. A treatment modality may be a specific drug treatment or may be a non-drug treatment such as cognitive behavioural therapy.

General purpose programming languages (GPPL) represent available computer architecture concepts as programs and are deliberately broad, so that they can be used to achieve almost any task that a programmer can conceive of. They offer this breadth by being “Turing Complete”, or “computationally universal”, which means they can approximately simulate the computational aspects of any other real-world general-purpose computer or computer language. Each GPPL offers a library of terms—a vocabulary—and rules about how those terms can be put together—syntax—which can be used to write general purpose programs with any computational objective. However, the breadth that GPPLs offer also introduces a risk of errors or bugs, either through incorrect translation of a programmer's idea into computer code, or through shortcomings in the design of the solution that the programmer has used to solve the problem or task at hand. In summary, GPPLs can be purposed to any scope of software task, but only if a correct code can be guaranteed (i.e., syntactically correct within its vocabulary, grammatically correct within its roles of use, functionally correct according to specified behavioural intent). However, the corresponding program would be brittle as it would work only for the specific case encoded with a focus on how to perform a given task.

Medical treatment decisions and pathways are inherently complex. Although a GPPL offers the breadth to represent the treatment complexity in software code, the complexity also creates risk that the corresponding code will contain errors. The breadth of the GPPL language can result in some errors only manifesting in specific uncommon scenarios and therefore going undiscovered during testing. However, once the GPPL software code is used by many people to deliver treatment, the risk that those low incidence errors can manifest, and cause harm increases significantly. Moreover, software bugs do not suffer wear and tear: if the circumstance for the bug manifests during program execution, the corresponding error will always occur.

The disclosed apparatus and methods can provide a means to demonstrate that specific errors cannot occur when a medical software program executes rather than relying on an inference from a set of potentially non-exhaustive test-cases that only covers a percentage of all possible cases (i.e., testing can only detect the presence of errors, whereas the disclosed apparatus can guarantee the absence of classes of errors through mathematical specification and verification of intent). In this way, the disclosed apparatus and methods provide a new level of safety for medical software programmes delivering actual treatment (“digital therapeutics”).

The disclosed apparatus and methods provide a Domain Specific Language (DSL) for precision medicine to impose safety guardrails during program compilation and to provide an intermediate executable program that ensures safeguards are met during program execution, when the program is executed by an execution engine in a suitable execution environment. Providing a declarative driven DSL with safety guardrails can provide a level of safety guarantee and reduce the risk of program errors in SaMD.

In some examples, the DSL provides a definition mechanism constrained by formal methods to ensure programs are well-formed (according to DSL syntax and DSL semantics) during validation and safety guardrails are enforced during execution. The DSL semantics can comprise a set of personalised medicine semantics that provide a limited set of declarative intents for a user (software developer). The declarative intents can comprise intents for safety guardrails, terminating conditions and/or treatment modulations. In this way, the DSL can build treatment packages according to user input to specify safety guardrails, terminating conditions and/or treatment modulations using formal methods. In addition, an execution engine may employ formal methods to enforce the safety guardrails, terminating conditions and/or treatment modulations (by creation of a run-time error) when executing the intermediate program in a suitable execution environment. The use of formal methods for the well-formedness checks during validation and the enforcement of run-time safety conditions can prevent specific errors from occurring and guarantee safety as desired. By applying formal methods to a bespoke DSL created to represent medical treatment decisions, it is possible to provide further guarantees that specific errors will never arise—such as a toxic dose threshold will never be crossed. However, it will be appreciated that the use of formal methods in ensuring well-formedness and/or guardrail enforcement is optional and that the disclosed apparatus and methods can provide an improved level of safety using alternative methods.

The DSL can be used to rapidly develop and deploy automated personalised medicine treatments, while the safety guardrails that may be enabled by formal methods provide assurance that the resulting treatment is safe with respect to desired properties, ensuring a solid basis for requesting regulatory approval for the digital treatment.

Regulatory bodies attempt to ensure that medical products that are made available to the public have assurance on their degree of safety and effectiveness. A methodology has evolved over decades to achieve this, e.g. around data validity, the double blind placebo controlled trial. These processes are lengthy and costly. Medical software can be challenging from a regulatory perspective because there is a desire for much more frequent updates, which don't fit neatly with the conventional approval processes. Furthermore, it can be hard for regulators to understand the detail within a software product and decide whether a change is allowable without repeating expensive studies. Software products encompassing artificial intelligence/machine learning can further complicate regulatory approval.

The disclosed apparatus and methods can provide personalised medicine treatment programs with an inherent level of safety that can provide a platform for regulatory assurance. As a result, regulators can carry out their regulatory function and understand what is being implemented with greater ease. Additionally, as discussed further below, the disclosed apparatus and methods can provide a platform suitable for an incremental regulatory process, whereby regulatory permission is given for the software device to operate within a defined set of operating parameters (eg physiological measurements). Within this approved operating range a personalised medicine treatment program may automatically modulate a treatment. However, following a deviation beyond the approved range or the creation of an error signal, the treatment program can terminate the automation and request clinician input.

Regulatory risk for software may be categorised as one of: Inform, Drive and Automate. Inform relates to the software processing data and presenting an output to a clinician, for example a moving average of blood pressure, or a sleep efficiency score. Drive relates to software providing a treatment change recommendation, wherein a clinician makes the final decision. Automate relates to software directly effecting change in the body (e.g. an implantable defibrillator) or directing the patient to change dose without clinician approval. The disclosed apparatus and methods can provide personalised medicine treatment programs that can provide Automate functionality within an approved regulatory range and Drive functionality outside of this range.

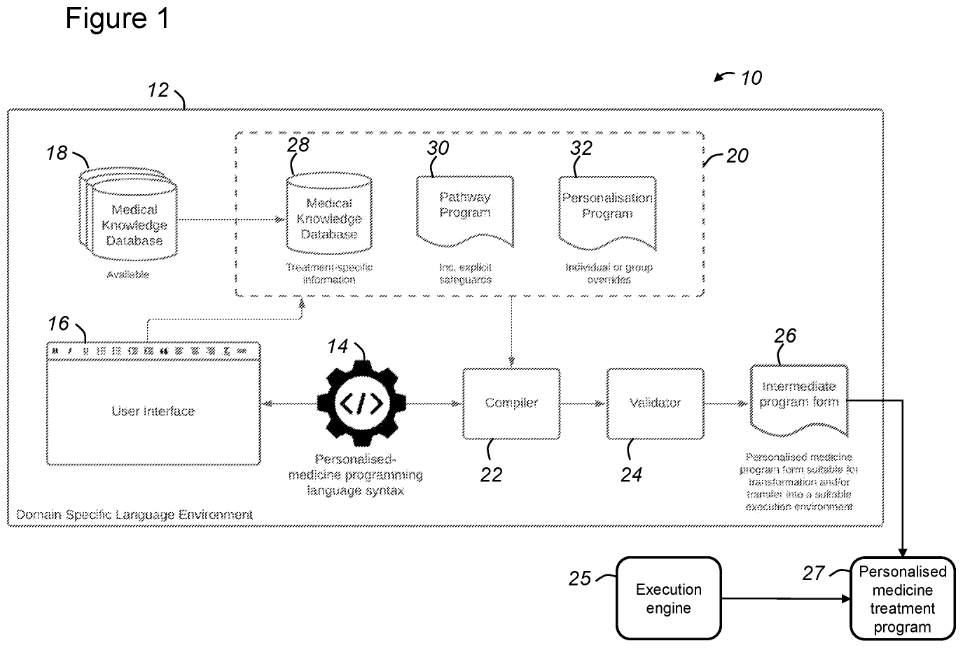

FIG. 1 illustrates an apparatus 10 according to an embodiment of the present disclosure. The apparatus 10 can produce an intermediate program form 26 or precursor of a personalised medicine treatment program 27. The personalised medicine treatment program 27 is a software program for delivering treatment to a patient. As explained below, the apparatus 10 enables the provision of the intermediate program form 26 in the DSL language such that the personalised medicine treatment program 27 can deliver automated personalised treatment with modulation of the treatment (dosage amount, dosage frequency etc) according to a patient's response in an inherently safe manner.

The apparatus 10 includes a domain specific language environment 12 comprising a personalised-medicine programming language syntax 14 (herein referred to as DSL syntax 14) and a user interface 16. The apparatus 10 has access to a medical knowledge database 18 which in this example resides within the apparatus 10 but in other examples may be accessed as a remote resource. The apparatus 10 can receive user input via the user interface 16, enabling a user (such as a medical software developer or programmer) to build a treatment package 20 for implementing one or more treatment modalities for a specific medical condition. The treatment package 20 is built using the DSL syntax 14 and data from the medical knowledge database 18 for the specific medical condition. The treatment package 20 can be structured in several ways, but generally includes: treatment objectives defining terminating conditions of the one or more treatment modalities; safety guardrails for ensuring patient safety; and treatment titration definitions for modulating the one or more treatment modalities. The apparatus 10 may further comprise a compiler 22 for compiling the treatment package 20 prior to validation. A validator 24 validates the treatment package 20 to demonstrate that the treatment objectives, safety guardrails and titration modulation definitions will be satisfied during execution. The validator 24 can output the validated treatment package as an intermediate program form 26.

The user interface 16 provides a tool for writing programs according to the domain specific language and conforming to the DSL syntax. The user interface 16 may comprise a graphical user interface for presentation on a ubiquitous computing device. In some examples the DSL environment 12 may be provided within an integrated development environment (IDE) and the user interface 16 may comprise a graphical user interface (GUI) of the IDE. The IDE may include one or more of: a code editor for entering code to define the treatment package 20; access to the medical knowledge database 18; the compiler 22; the validator 24; and a debugger for debugging the code according to the DSL syntax 14 and DSL semantics.

In this example, the treatment package 20 comprises a treatment knowledge database 28, a pathway program 30 and a personalisation program 32.

The treatment knowledge database 28 comprises data from the medical knowledge database 18 relating to the specific medical condition of the treatment package 20. In other words, the treatment knowledge database 28 comprises a selected subset of the medical knowledge database 18.

The pathway program 30 is written in the DSL syntax and describes a specific medicine treatment pathway for a medical condition. The pathway program 30 can include measurements, doses, treatment titrations, treatment ambitions, and treatment objectives relating to the specific medical condition. The Pathway program may express the safety guardrails in the form of treatment objectives (termination conditions) and treatment ambitions (invariant properties).

The personalisation program 32 is written in the DSL syntax and can describe one or more overrides to the pathway program to personalise it for an individual or for a group of patients.

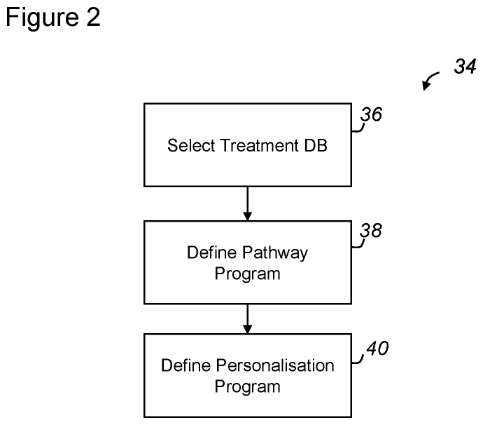

The treatment package 20 and its subcomponents 28, 30, 32 are described in further detail below in relation to FIG. 2.

The compiler 22 can process the treatment package 20 to:
i. expand implicitly-defined/derived syntactic and semantic constructs to improve usability, decrease complexity and enforce consistency. Specifically, this activity may focus on introducing implicit titration expressions and implicit safety guardrails.
ii. Infer and override information from linked programs.

The validator 24 can perform well-formedness checks against semantic criteria, to prevent ill-formed programs, and various program inference and overriding rules. The validator 24 can output the intermediate program form 26. The Intermediate Program Form 26 is a representation of the expanded, validated treatment package 20. The intermediate program form 26 may be considered as a precursor to the personalised medicine treatment program 27 that will operate on a patient's (end-user's) device and deliver the personalised treatment. The intermediate program form 26 may have explicit safety guardrails (defined by the user input) and implicit safety guardrails (introduced by the compiler 22), treatment objectives and titration modulation criteria.

The intermediate program form 26 may be combined with an execution engine 25 to form the personalised medicine treatment program 27. The execution engine 25 may include code for executing the intermediate program form 26 and monitoring a program state such that the safety guardrails, treatment objectives and titration modulation criteria are safely and accurately enforced.

The deployment of the personalised medicine treatment program 27 may occur in a number of ways. For example, the execution engine 25 and intermediate program form 26 may be compiled into an executable personalised medicine treatment program 27 to run on a target operating system. As a further example, the intermediate program form 26 may be deployed onto a previously installed execution engine 25 that runs on a target operating system. As a yet further example, the intermediate program form 26 and execution engine 25 may be deployed and installed together at the same time to provide the personalised medicine treatment program 27. The intermediate program form 26 may undergo one or more further transformations prior to deployment (e.g. optimisation for speed, platform specific optimisations etc).

Defining the Treatment Package—User Input

FIGS. 2 to 5 illustrate methods of receiving the user input to build the treatment package 20 according to embodiments of the present disclosure. The methods will be described as performed by the apparatus 10 of FIG. 1 receiving user input at the user interface 16 to define the treatment package 20.

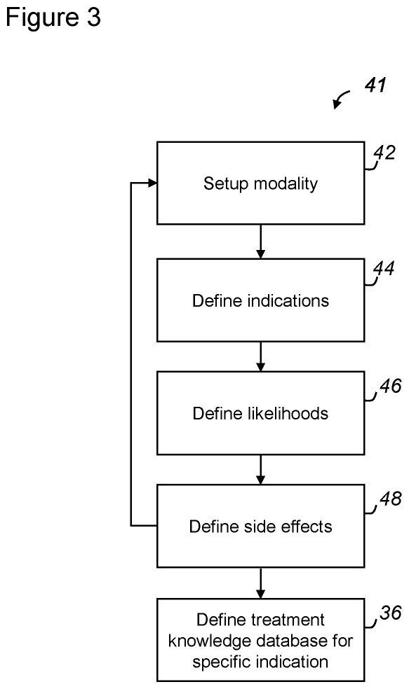

FIG. 2 illustrates a method of receiving user input to build the treatment package according to an embodiment of the present disclosure. A first step 36 comprises receiving user input to select the treatment knowledge database 28. In some examples the treatment knowledge database 28 may be predefined and may be a sub-set of a larger medical knowledge database 18, as described below with reference to FIG. 3. In other examples, step 36 may comprise defining the treatment database. The treatment knowledge database 28 comprises trusted information on one or more treatment modalities for treating the specific medical condition of the treatment package 20. The trusted information comprises characteristics and constraints of each of the one or more treatment modalities from a trusted source, for example the BNF, NICE guidelines etc. The one or more treatment modalities may include a database of drugs regulated for use with the specific medical condition. The characteristics and constraints may include safe dosage levels and side effects associated with each treatment modality.

A second step 38 comprises receiving user input to define the pathway program 30. The pathway program 30 can define the main treatment algorithm for delivering the treatment for the specific medical condition in the personalised medicine treatment program 27. The pathway program 30 may be considered to “beat” during execution of the personalised medicine treatment program 27. That is, the code defined in the pathway program 30 executes at the beat rate which is the desired frequency of treatment (daily, 12-hourly, etc). The concept of program “beats” is discussed further below.

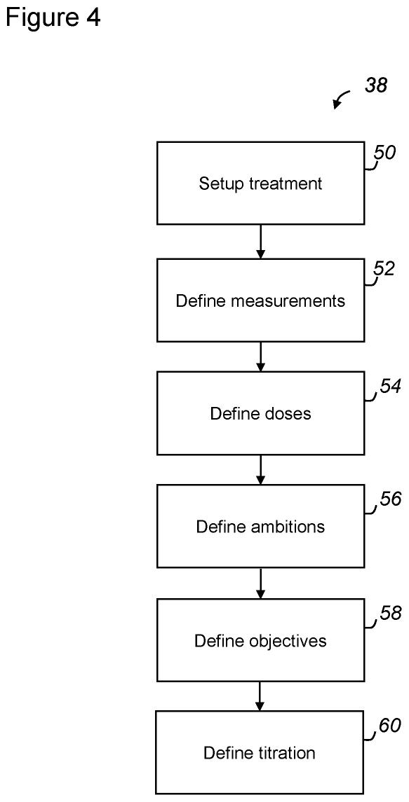

User input is received to define a set of sub-components of the pathway program 30. The sub-components can comprise any of: (i) patient measurements for the one or more treatment modalities; (ii) dosages for the one or more treatment modalities; (iii) treatment objectives defining terminating conditions for the one or more treatment modalities; (iv) treatment ambitions defining one or more invariant properties to be (desirably or mandatorily) satisfied throughout the treatment for the one or more treatment modalities; and (v) treatment titration definitions for modulating the one or more treatment modalities as the treatment is delivered. These sub-components are described in further detail below in relation to FIG. 4.

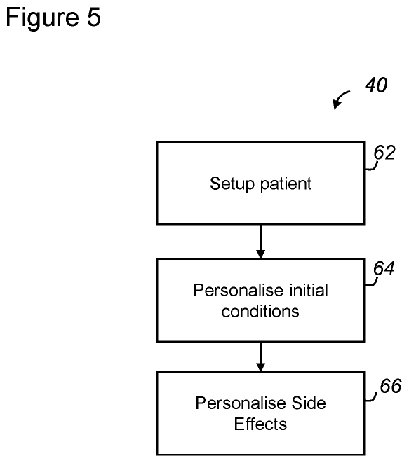

A third step 40 comprises receiving user input to define the personalisation program 32. The personalisation program 32 describes personalised overrides to be made to the pathway program 30. Definition of the personalisation program is discussed further below in relation to FIG. 5.

FIG. 3 illustrates a method of defining a treatment knowledge database 28 according to an embodiment of the present disclosure. In this example, a medical knowledge database 18 is defined at steps 42 to 48 followed by selection (step 36) of a subset of the medical knowledge database 18 as the treatment knowledge database 28. It will be appreciated that steps 42 to 48 may be performed once to define a global medical knowledge database 18 for a plurality of personalised medicine treatment programs. As a result, it will be further appreciated that the method of producing an intermediate program form 26 may comprise step 36 and not steps 42 to 48. It will further be appreciated that in other examples (not illustrated), a treatment knowledge database 28 may be defined directly according to steps 42 to 48 but limited to the specific medical condition of the treatment knowledge database 28.

Steps 42 to 48 comprise translating a trusted medical source into a logical form understood by the DSL environment 12. In some examples, the medical knowledge database 18 and the treatment knowledge database 28 may be defined using the DSL syntax 14.

A first step 42 comprises receiving user input to define a first modality (a drug, for example). The user input may also define a beat time unit reference (e.g., hourly, daily etc.). The time unit reference does not necessarily correspond to a modality delivery frequency but a unit of time that such a frequency can be expressed in terms of. The concept of program “beats” is discussed further below.

A second step 44 comprises receiving user input to define all medical conditions treated by the first modality. For each medical condition, the user input may define characteristics associated with the first medical condition for example, dosage unit, frequency, amount, toxic frequency, toxic amount, regulatory approved amount, regulatory approved frequency etc. An example DSL program syntax, with DSL keywords in bold, is:

```
bnf Amlodipine beating hourly {
  bnf_{—}conditions {
  Hypertension: ug, 24, 2000, 24, 10000;
  Angina : ug, 24, 5000, 24, 10000;
}
```

defining a modality (the drug Amlodipine), a reference time unit of hours (beating hourly), two medical conditions (Hypertension and Angina), dosage unit (micrograms, ug), dosage frequency (24 hours), dosage amount (2000 ug for Hypertension, 5000 ug for Angina), toxic frequency (24 hours) and a toxic amount (10000 ug). As described herein, a toxic dose (amount/frequency) may refer to an upper bound dosage that the treatment program can output/recommend to a patient or clinician. The term toxic does not necessarily relate to a toxic level that would cause severe side-effects or organ damage. A regulatory approved frequency/amount may refer to a dosage range within which automated personalised treatment has received regulatory approval, and outside which the personalised medicine treatment program 27 must operate in a drive or inform mode (see description of inform, drive automate above).

A third step 46 comprises receiving user input to define treatment modality side effect likelihoods of occurrence according to medical-specific vocabulary (e.g., common, rare, very common, etc.). This maps the vocabulary in the trusted data source to a (statistically) expected chance of occurrence thereby quantifying the vocabulary terms (e.g., fatigue as a “common” side effect for the amlodipine drug when treating hypertension and may be assigned a corresponding likelihood index).

A fourth step 48 comprises receiving user input to define modality-specific side effects of interest, whether they are to be measured, and their likelihood of occurrence according to the fixed likelihood vocabulary defined at step 46 (i.e., links side effects with their medical-specific expected chance of occurrence).

Steps 42 to 48 can be repeated for all treatment modalities to be translated from the trusted source.

Step 36 comprises selecting a subset of the medical knowledge database 18 created by steps 42 to 46 , to define the treatment knowledge database 28 for the specific medical condition to be treated by the personalised medicine treatment program 27.

In some examples, the medical knowledge database 18 and/or the treatment knowledge database 28 may be written in the DSL. In other examples, one or both may form part of the compilation process either from a DSL script in a self-managed document storage, or from a trusted third party source (e.g. BNF/NICE, which have their own medical database/access.) The latter can ensure that the treatment knowledge database 28 includes the most up to date data.

FIG. 4 illustrates a method 38 of defining the pathway program 30 according to an embodiment of the present disclosure.

A first step 50 comprises receiving user input to select the specific medical condition for the treatment package 20 (and ultimately the personalised medicine treatment program 27). The user input may also comprise a beat time unit reference—the pathway execution beat—defining how often the pathway program will execute during the treatment (e.g., daily, 12 hourly etc). The concept of program “beats” is discussed further below. An example DSL program syntax representing the first step may take the form:

```
pathway Hypertension for PatientX
      from “2020-10-10 08:00 BST” beating daily
    {
  ...
}
```

A second step 52 comprises receiving user input to define measurements required for the treatment. The user input may define physical measurement inputs (for example blood pressure readings, heart rate etc.) and/or logical measurement inputs (for example, side effect severity ratings from the patient) associated with the treatment. The user input may define the measurements in DSL syntax as “name: unit, frequency, ranges, . . . ” (see example code below, lines 3 to 27). As discussed below, the personalised medicine treatment program 27 comprises a treatment state defining a current program state (or sigma) of the program when running in the end-use execution environment. The treatment state can comprise among other items, a history of measurements taken at the appropriate program beat.

A third step 54 comprises receiving user input to define one or more (initial) doses of the one or more treatment modalities for the specific medical condition. The user input may define the treatment modality by reference to the treatment knowledge database 28 using a modality link. The link to the treatment knowledge database 28 can import the medical knowledge and safety data for each treatment modality (e.g., drugs) for the specific medical conditions. As discussed further below, the apparatus 10 can define implicit safety guardrails during compilation of the treatment package 20. The apparatus 10 may use the trusted data from the treatment knowledge database 28 when defining one or more of these implicit safety guardrails. The user input may define the dosage of each treatment modality in DSL syntax as “name: modality-link, initial amount, unit, frequency”.

doses(any) {d_amlodipine: Amlodipine, 4, mg, daily;}

A fourth step 56 comprises receiving user input to define one or more explicit treatment ambitions. Treatment ambitions define one or more invariant properties to be satisfied throughout the treatment for the one or more treatment modalities. As discussed further below, treatment ambitions may be desirable or mandatory. The treatment ambitions can be assessed for every beat of the treatment (or every execution step of the pathway program). As discussed below, the personalised medicine treatment program 27 comprises a treatment state defining a current program state (or sigma) of the program when running in the end-use execution environment. The treatment ambitions may comprise expressions over the treatment state, per beat, throughout treatment. The accumulated treatment state differential can then form a constituent part of a certificate of successful treatment.

The treatment ambitions may comprise a predicate to remain true throughout the treatment delivered by the personalised medicine treatment program 27.

Treatment ambitions defined from user input may define explicit (user defined) safety guardrails that ensure patient safety during treatment. Such safety guardrails may be mandatory conditions, such that the execution engine 25 will create an error if the treatment ambition returns a false value and the personalised medical treatment program 27 may terminate. The execution environment (or execution engine 25) may terminate the personalised medical treatment program 27 in response to the error. Explicit treatment ambitions may also comprise desirable treatment ambitions, such that the execution engine may institute a warning rather than an error if the treatment ambition returns a false value. The personalised medical treatment program 27 may respond to the warning in a number of ways, for example, log the warning, output the warning to the patient or clinician etc. An example syntax for a desirable explicit treatment ambition for no high degree of side effects can be written as:

```
ambitions (all) {
  no_high_se(desirable):
    side_effects_for_are(all, all, all, high, below);
}
```

The ambition has a mathematical predicate defined as a parameterised function named “sife_effects_for_are”, which may be defined in a separate part of code (see for example, the program definitions section in the example code below, line 64). The function defines a complex logical predicate ensuring that all measurements for all side effects are below any value range (see example code, lines 21 . . . 26) rated as high.

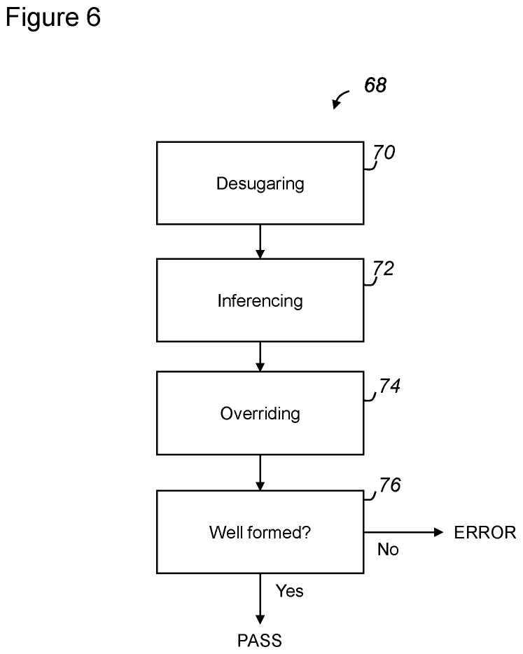

As described below in relation to FIG. 6, implicit treatment ambitions (an example of implicit safety guardrails) may also be defined during compilation of the treatment package 20 to form the intermediate program form 26. The treatment ambitions/mathematical predicates may be defined using one or more formal methods.

A fifth step 58 comprises receiving user input to define one or more explicit treatment objectives. Treatment objectives define terminating conditions for the treatment delivered by the personalised medicine treatment program 27. A treatment objective may comprise a predicate, for monitoring by the execution engine 25, that once true will terminate the treatment program 27. The treatment objectives may comprise expressions over the treatment state per beat throughout treatment. The predicate may comprise one or more formal methods. The treatment objectives may be either normal (i.e., completion of treatment course, whether successful or ineffective) or adverse (i.e., early termination of the treatment due to adverse reaction or error).

In a similar way to treatment ambitions, explicit treatment objectives defined by the received user input may define explicit (user-defined) safety guardrails for ensuring patient safety. For example, an explicit treatment objective may define a safety guardrail by defining that any adverse side effects encountered during the treatment will terminate the treatment. An example of such code written in a DSL syntax is:

```
  objectives (any) {
    adverse: side_effects_for_are(any, any, any, adverse,
within);
  }
```

As described below in relation to FIG. 6, the apparatus 10 of FIG. 1, specifically the compiler 22, can also define implicit treatment objectives (an example of implicit safety guardrails) during compilation of the user-defined treatment package 20. The explicit and implicit safety guardrails provide the inherent safety guarantee for the personalised medicine treatment program 27.

A sixth step 60 comprises receiving user input to define one or more (explicit) treatment titration definitions for modulating the one or more treatment modalities. The treatment titration definitions (herein referred to as titrations, titration criteria, treatment titration criteria and the like) may comprise criteria expressions that modulate the treatment. The criteria expressions may comprise one or more logical predicates. Modulation of the treatment may comprise modulation of measurement frequency, dose frequency, dose amount and/or any other element of the treatment over the course of the treatment. The titrations can influence the treatment trajectory (in combination with other parts of the pathway program). Titration modulation during treatment program execution may comprise the execution engine 25 evaluating criteria expressions over program states before and after intervention, hence determining how treatment trajectory can change. As an example, an explicit treatment titration definition may define how a drug dosage amount can be modulated during the course of treatment based on measured side-effects. An example of such code written in a DSL syntax is:

| titration (optimise) { | |
|---|---|
| criteria(any,side_{—}effects,dose=d_amlodipine,frequency=nil, | |
| window=to_{—}week(1)) | { |
| baseline | (window=to_{—}week(2), amount=+0); |
| acceptable | (modifier=all, amount=+2); |
| uncomfortable | (amount=+2); |
| intolerable | (amount=−2); |
| adverse | (amount=0); |
| } | |
| objectives (any) | { stable; adverse; } |

}

As illustrated by the example code, the treatment titration definitions define modulations to the treatment such as adjustments to dose (up or down) based on observed measurements or other treatment state. In this example drug doses amount is adjusted either up or down based on observed side effects.

Further, treatment titration definitions have objectives that define terminating conditions for the treatment modulation. In this way, treatment modulations can operate (or not) at various stages throughout treatment.

In the above example, the titration has the same objectives of the overall pathway program, which defines a stable objective (blood pressure stable for 4 weeks) and an adverse objective (any adverse side effect). As a result, in this example, termination of the treatment modulation by the execution engine 25 will correspond to termination of the whole personalised medicine treatment program 27.

In some examples, treatment modulation may only operate after some initial treatment time (i.e., number of beats) has passed, so that a treatment can take effect before any intervention (see sample code window=to_week(1)). In this way, treatment modulation may operate at a different beat (a titration beat frequency) than the treatment program 27 (e.g., treatment program beats daily, but titrations evaluated weekly). This corresponds to the way treatment modulation is delivered conventionally: a patient sees their doctors to assess, and possibly change, direction of treatment less frequently than they undertake a treatment.

The execution engine 25 may evaluate and resolve titration criteria during execution. The evaluation and resolution of the criteria can define the treatment trajectory. The treatment package 20/personalised medicine treatment program 27 may have multiple titration criteria and titration objectives. This can provide expressive treatment choices. For example, multiple criteria for a hypertension treatment program may include criteria to modulate Amlodipine dosage amount/frequency up or down, depending on how multiple (personalised) side-effects evolve over time.

As multiple criteria may create treatment “competition”, where one criterion takes treatment in one direction, whereas another might take it in the opposite direction, the execution engine 25 may perform titration criteria resolution during execution. By performing criteria resolution, the execution engine 25 can determine which criteria takes priority in terms of keeping all pathway safety guardrail treatment ambitions and treatment objectives satisfied (i.e., criteria that break mandatory ambitions or objectives are ranked lower), and/or terminating a specific titration modulation. In some examples, during resolution, all criteria may be evaluated/attempted, yet only those resulting in no failures or those with minimal faults are accepted. The criteria resolution should be unique (i.e., there are no two satisfiable criteria at the same time) to avoid non-deterministic choices within the program execution. In some examples, the validator 24 may only validate treatment packages 20 with a unique titration criteria resolution as part of the well-formedness checks. Resolution-driven titration-criteria evaluation can advantageously cater for multiple treatment scenarios without becoming “brittle” (i.e., only work in a narrow field of options/conditions) like a GPPL-defined treatment program.

Titration criteria may be semantic linked with other components of the treatment program 27. For example, the titration criteria may refer to measurements and doses. Additionally, titration objectives can be semantically linked with the treatment objectives (see example code above). In some examples, this may result in treatment terminating when titration terminates, or else if neither have been achieved after a specified time.

As described above, titration criteria expressions may operate on a different beat to the pathway. The execution engine 25 may resolve the difference in the relative time associated with how different program parts beat (e.g., doses every 8 hours, measurements every 16 hrs, whereas the treatment program beats daily), whilst deciding which titration criteria expression holds for the current program state.

The combination of measurements over time and the one or more treatment titrations of the pathway program 30 can provide personalisation to the personalised medicine treatment program 27. This is because the titrations can modulate the treatment based on the measured response of an individual patient over time. The explicit and implicit safety guardrails provided by the treatment objectives and ambitions ensure that this modulation of treatment is carried out in an inherently safe manner (i.e., if they fail by evaluating to FALSE, treatment stops). The personalisation module 32 provides a further optional level of patient personalisation to the personalised medicine treatment program 27.

FIG. 5 illustrates a method 40 of defining the (optional) personalisation program 32 according to an embodiment of the present disclosure.

A first step 62 comprises receiving user input to define a patient identifier or a patient group identifier (e.g. based on age, sex, BMI etc) and a personalisation start time. The patient identifier enables the personalisation program 32 for the specific patient or group to be called from or linked to the pathway program 30. Program linking can enable patient customisable options defined in the personalisation program 32 to override pathway program 30 definitions, such as for personalised side effect tolerances. The patient customisable options (including personalised initial conditions and personalised side effect tolerances, discussed below) may comprise a constrained set of options that can be selected by a clinician or patient prior to execution of the treatment program 27 (e.g. during installation of the program 27).

A second step 64 comprises receiving user input to define one or more patient customisable options defining personalised initial conditions. The personalised initial conditions may include patient-specific initial measurements and/or dose information. As explained below, during compilation, the personalised initial conditions may override the corresponding definitions in a linked pathway program 30.

A third step 66 comprises receiving user input to define one or more patient customisable options for personalised side effect tolerances. The personalised side effect tolerances may define a particular tolerance (or lack of) to a specific side effect. For example, a patient may accept a higher level of fatigue than a default acceptable level of fatigue. The default acceptable level of a side effect may be defined by the linked default side effect likelihood in the treatment-specific medical knowledge database 28. As with the personalised initial conditions, the personalised side effect tolerances may override the corresponding definitions in a linked pathway program 30 during compilation.

The processes described in relation to FIGS. 2 to 5 comprise collecting information in various stages, each of which influence treatment needs, treatment trajectory, and ultimately, treatment outcomes. The process can start by providing (or referring to) available medical knowledge 36, where information about different drug posologies (steps 42 and 44) and side effects (steps 46 and 48) are characterised. Next, a specific treatment pathway (step 50) characterises the DSL's core concepts (step 38): input measurement type and frequency (e.g., blood pressure daily) (step 52); chosen drug dosage (step 54), which can be linked with the treatment knowledge database 28 (e.g., 2 mg of Amlodipine daily to treat hypertension); treatment ambitions (step 56), which are enforced throughout treatment (e.g., no uncomfortable side effect); treatment objectives/termination conditions (step 58); and treatment titration (or modulation) conditions step (60), which determine the treatment trajectory (e.g., change in dose or measurement quantity and/or frequency over time depending on specific criteria). Finally, an optional patient personalisation (step 40) may be defined to refine the pathway program 30, to take patient or patient-group specific-dose conditions (step 64) and side effects (re-) interpretation according to personalised tolerances (step 66) into account.

An example DSL program syntax for the specific medical condition hypertension, encompassing all the steps (36, 38, 40) of FIG. 2 may be written (with DSL keywords in bold) as:

| | |
|---|---|
| 1 | pathway Hypertension for PatientX |

```
      from “2020-10-10
      08:00 BST”
```

beating daily {

| 2 | | |
|---|---|---|
| 3 | measurements(non_{—}empty,all) { | |
| 4 | Systolic_BP: mmHg, daily, | |
| 5 | { “death” | −> low: <60, |
| 6 | “low” | −> low: 60..89, |
| 7 | acceptable | −> okay: 90..120, |
| 8 | “elevated” | −> okay: 121..129, |
| 9 | “hyper1” | −> high: 130..139, |
| 10 | “hyper2” | −> high: 140..179, |
| 11 | adverse | −> high: >=180 }; |
| 12 | | |
| 13 | Diastolic_BP: mmHg, daily, | |
| 14 | { “death” | −> low: <50, |
| 15 | “low” | −> low: 50..59, |
| 16 | acceptable | −> okay: 60..80, |
| 17 | “hyper1” | −> high: 81..89, |
| 18 | “hyper2” | −> high: 90..119, |
| 19 | adverse | −> high: >=120 }; |
| 20 | | |
| 21 | side_{—}effects(criteria, daily) | |
| 22 | { acceptable | −> okay: 1..1, |
| 23 | uncomfortable | −> high: 2..2, |
| 24 | intolerable | −> high: 3..3, |
| 25 | adverse | −> high: 4..4 } |

| | |
|---|---|
| 26 | { Oedema; Fatigue; Dizziness; } |
| 27 | } |
| 28 | |
| 29 | doses (any) { d_amlodipine: Amlodipine, 4, mg, daily; |

}

| | |
|---|---|
| 30 | |
| 31 | objectives (any) { |
| 32 | o_min_12w: has_been_measured_for(measurements, 12, |

to_{—}week);

| | |
|---|---|
| 33 | |
| 34 | stable: |
| 35 | stable_over_at_for(Systolic_BP , 4, to_{—}week, |

120) and

| | |
|---|---|
| 36 | stable_over_at_for(Diastolic_BP, 4, to_{—}week, |

80);

| | |
|---|---|
| 37 | |
| 38 | adverse: |

side_effects_for_are(any,any,any,adverse,within);

| | |
|---|---|
| 39 | } |
| 40 | |
| 41 | ambitions (all) { |
| 42 | no_high_se(desirable): side_effects_for_are(all, |

all, all, high, below);

| | |
|---|---|
| 43 | |
| 44 | expected_bp_delta(desirable): |
| 45 | expected_delta(Systolic_BP, |
| 46 | {2 \|−> 5.1%, 4 \|−> 8.0%, 6 \|−> 11.1%, 8 \|−> |

| | |
|---|---|
| 12.4%, | 10 \|−> 13.4%}) |

| | |
|---|---|
| 47 | and |
| 48 | expected_delta(Diastolic_BP, |
| 49 | {2 \|−> 5.8%, 4 \|−> 7.7%, 6 \|−> 9.2%, 8 \|−> |

| | |
|---|---|
| 9.7%, | 10 \|−> 10.0%}); |

| | |
|---|---|
| 50 | } |
| 51 | |
| 52 | titration (optimise) { |
| 53 | |

criteria(any,side_{—}effects,dose=d_amlodipine,frequency=nil,
window=to_{—}week(1)) {

| | | |
|---|---|---|
| 54 | baseline | (window=to_{—}week(2), amount=+0); |

| 55 | acceptable | (modifier=all, amount=+2); |
|---|---|---|
| 56 | uncomfortable | (amount=+2); |
| 57 | intolerable | (amount=−2); |
| 58 | adverse | (amount=0); |
| 59 | } | |
| 60 | | |
| 61 | objectives (any) | { stable; adverse; } |

| | |
|---|---|
| 62 | } |
| 63 | |
| 64 | definitions { ... } |
| 65 | } |
| 66 | |
| 67 | patient PatientX from “2020-10-11 08:41 BST” { |
| 68 | initial_{—}dose { Amlodipine: 2, mg, daily } |
| 69 | |
| 70 | side_{—}effects(criteria,daily) ++ {uncomfortable \|−> |

okay: 2..2}

| | |
|---|---|
| 71 | { Fatigue; Dizziness; } |
| 72 | } |
| 73 | |
| 74 | bnf Amlodipine beating hourly { |
| 75 | bnf_{—}conditions { |
| 76 | Hypertension: ug, 24, 2000, 24, 10000; |
| 77 | Angina : ug, 24, 5000, 24, 10000; |
| 78 | } |
| 79 | |
| 80 | bnf_{—}likelihood { ... } |
| 81 | bnf_{—}effects { ... } |
| 82 | } |

Following receipt of the user input, the apparatus may perform compilation and validation on the treatment package 20. Compilation and validation may also be performed following one or more of the individual processes 34, 36, 38 and 40.

### Defining the Treatment Package—Compilation and Validation

A compilation and validation process may be appended to any of the process steps of FIG. 2. The following description describes performance of a compilation and validation process following completion of the process of FIG. 2 and the user definition of the treatment package 20. However, it will be appreciated that the described process can also be applied following definition of any of the treatment knowledge database 28, the pathway program 30 and the personalisation program 32. As described below, the compilation and validation process plays an important role in the inherent safety of the personalised medicine treatment program 27 by unwrapping and defining implicit safety guardrails and performing well-formedness checks, both of which can be defined using formal methods such as mathematical logical predicates.

FIG. 6 illustrates a compilation and validation process 68 according to an embodiment of the present disclosure. The first three steps 70, 72, 74 may be considered as the compilation process and the fourth step 76 may be considered as the validation process.

A first step 70 comprises desugaring the treatment package 20. This can comprise expanding implicitly-defined syntactic and/or semantic constructs. The expansion can improve usability, decrease complexity and/or enforce consistency, and minimise code maintenance.

In the example code snippets provided above, user input is constrained to inputting declarative intents using the DSL syntax. The DSL may comprise further intrinsic code that underpins the declarative intents and cannot be edited by the user (treatment program developer). Desugaring may comprise expanding out such intrinsic code. In this way, the provision of the DSL deskills the treatment program development process and can reduce (or even prevent) the definition of errors by the user.

A user can change the underlying complex logical meaning of a predicate by simply varying the function call parameters (all, all, low, above in the expression above). However, the burden of specification of such complex logical predicates is low for the user. This is achieved through careful definition of the intrinsic code such as parameterisation of concepts, and sufficient encapsulation through semantic function abstractions, as mathematical logical predicate definitions (see line 64 in the code above).

In addition, stringent regulatory approval of the DSL can be performed once and any subsequent treatment programs defined by a user using the DSL may require a less stringent or laborious approval process because performance of the well-formedness checks and implicit safety guardrails have already been approved for general/universal use.

A second step 72 comprises inferring information from linked programs. For example, the pathway program 30 can import trusted information from the treatment knowledge database 28, such as toxic dose levels, adverse side effects etc. Treatment outcomes can vary depending on the linked program being inferred.

A third step 74 comprises overriding information from linked programs. For example, side effect measurements defined (or inferred) in the pathway program 30 can be overridden by a linked personalisation program 32 (patient choice takes precedence over pathway choice). Treatment outcomes can vary depending on the linked program being overridden.

A fourth step 76 comprises performing well-formedness checks against semantic criteria. The validator 24 may perform well-formedness checks on the treatment package 20 using formal methods or any other suitable approach as known in the art. Any failures will result in a validation error. If all well-formedness checks are passed, the validator 24 may output the validated treatment package 20 as the intermediate program form 26.

### Implicit Safety Guardrails and Titration Expressions

For a user-input defined DSL pathway program 32, the compiler 22 can automatically define implicit safety guardrail ambitions and objectives during the compilation process, particularly during desugaring 70 and program inference 72. This implicit program “wrapping” can enforce a series of implicit safety guardrails for all intermediate program forms 26 and personalised medicine treatment programs 27 without any user intervention. Indeed, the DSL environment 12 is designed to introduce such implicit safety guardrails by default and users cannot avoid or overrule their introduction. For example, a user cannot define titration criteria or a safety ambition that would betray medical knowledge toxicity boundaries for a drug (or modality) used in the treatment. As a result, it is impossible for the user to define a treatment program 27 that could overdose by mistake, as the resulting user defined code would entail at least one of: (i) an ill-formed program that would be caught during program validation 76; or (ii) an inconsistent implicit treatment ambition that would be caught (result in error and treatment termination) during execution of the treatment program 27. As a further safety net, the compiler may define an implicit treatment objective requiring immediate termination of the treatment if an adverse side effect occurs at any beat of the treatment program execution.

During the desugaring 70 and/or inferring 72 information from linked programs steps, the compiler 22 can introduce one or more implicit safety guardrails as implicit treatment objectives and/or implicit treatment ambitions. Such implicit safety guardrails may comprise mandatory safety guardrails that lead to the termination of treatment if breached (see discussion below on mandates). The treatment ambitions may comprise a logical predicate that must (mandatory) or should (desirable) remain true during execution of the treatment program 27. The treatment objectives may comprise a logical predicate that will terminate the treatment if it becomes true.

As a first example, the compiler 22 can introduce one or more implicit toxicity safety guardrails, corresponding to the one or more treatment modalities, during the inferring step 72. The compiler 22 may infer each implicit toxicity safety guardrail as an implicit treatment ambition based on medically known levels of toxicity for the modality as defined in the linked treatment knowledge database 28. The one or more implicit toxicity safety guardrails can ensure that no treatment program 27 using a particular drug can ever prescribe or measure beyond toxic amounts over toxic frequencies.

As a second example, the compiler 22 can introduce an implicit sensor safety guardrail for each sensor-based measurement defined in the pathway program (step 52), during the desugaring step 70. The compiler 22 may define each implicit sensor safety guardrail as an implicit treatment ambition. The example hypertension program DSL syntax defined above is used to demonstrate definition of the implicit sensor safety guardrail. Lines 4 to 11 of the DSL syntax code define seven systolic blood pressure ranges. Each range has an associated rating of low, okay or high. The compiler can define the implicit sensor safety guardrail to safeguard against sensor measurements rated low. In this example, low corresponds to a systolic measurement of less than 89 (60-89 is dangerously low, whereas less than 60 can be considered as near death—see DSL syntax code lines 5 and 6). The compiler 22 can define the implicit sensor safety guardrail as an implicit treatment ambition. An example DSL syntax may be written:

a_SysBP_low(mandatory):
measurement_within_bounds_for(all, all, low, above);

In this example, four parameters in the function call provide modifiers to simplify the use of the underlying complex logical predicate. The predicate checks that for all (1) measurement amounts taken so far (during the treatment), and for all (2) measurement (3) ranges having a low rating, the measured amounts are strictly (4) above the highest value of the low rated ranges. The example sensor safety guardrail can avoid both faulty blood pressure monitors or treating patients that have died or are with severely low (albeit accurate) blood pressure readings. If either of these scenarios occurs (measurement less than 90) the treatment ambition evaluation will not be satisfied, treatment tracing will update the treatment state, and the treatment will terminate because the treatment ambition is mandatory. In this example, the compiler 22 would also define an implicit sensor safety guardrail for the diastolic blood pressure measurement (see lines 13 to 19 of example DSL program syntax). In some examples, the compiler 22 may also define an implicit sensor safety guardrail for a measured dose for treatments in which a user measures dosages (following modulation of a dosage amount by a titration definition).

The compiler 22 may define other implicit safety guardrails ambitions and objectives. Implicit guardrails may include implicit side effect safety guardrails. In some examples, the implicit side effect safety guardrails may comprise a mandatory treatment ambition or objective, for example adverse side effects terminate the treatment. In some examples, implicit side effect safety guardrails may comprise desirable treatment ambitions.

The compiler 22 may introduce implicit safety guardrails with a desirable mandate during the overriding step 74 by importing personalised side effect tolerances from the personalisation program 32. For example, the patient personalisation program 32 may define a preferred side effect tolerance, such as all side effect measurement should be within a low range. The compiler can import this preference into the treatment package 20 as a desirable side effect treatment ambition. The desirable side effect treatment ambition is not mandatory, particularly because side effect variation is expected. Breach of the desirable side effect treatment ambition may not terminate the treatment. As discussed above, the treatment titration expressions/criteria may include an instruction to reduce a dosage level of the treatment modality if the desirable side effect treatment ambition is breached. In this way, the side effect can be modulated and mitigated through the personalisation program 32 link to the pathway program 30.

In some examples, an implicit treatment objective or an implicit mandatory treatment ambition may be based on side effect combinations for detecting an overdose. For example, the treatment objective or ambition may define a predicate based on a side effect combination indicative of a treatment modality overdose. The execution engine 25 may then terminate the treatment program 27 based on a value of the predicate indicating the side effect combination has been measured. The compiler 22 may infer the side effect combinations indicative of an overdose from the treatment knowledge database 28. In this way, the personalised medicine treatment program 27 can detect accidental patient overdoses, terminate the program and generate an alarm when a patient mistakenly takes a higher dose than that prescribed by the clinician or program 27.

In some examples, the implicit safety guardrails are (power-) user configurable. In some examples, extreme circumstances may determine what is suitable. For example, cancer treatment may genuinely overshoot drug toxicity levels or adverse side effects.

The compiler 22 introduces the implicit safety guardrails introduced into the treatment package 20 during the compilation process 70-74. The validator can check the implicit safety guardrails for well-formedness (step 76) as a further check on the well-formedness of the user input. For example, an ill-formed explicit treatment ambition may be identified because it is inconsistent with an implicit treatment ambition.

As touched on above, the treatment objectives and ambitions (both explicit and implicit) may refer to or call a treatment state (a program state or sigma) of the personalised medicine treatment program 27 over time. In this way, the treatment objectives and ambitions can comprise delayed computation structures for guaranteeing that safety guardrails and titration modulation predicates remain true at every program state for all program states executed. As a result, the execution engine 25 can check the treatment objectives and ambitions as part of a ‘treat’ step for each beat of the personalised medicine treatment program 27. To expand on this, we first describe further the execution of the personalised medicine treatment program 27.

In the same way as described above in relation to implicit safety guardrails, the compiler 22 can also introduce implicit titration expressions during the desugaring and inferring steps 70, 72.

### Execution

A computer program can store data in variables, which represent storage locations in the computer's memory. The contents of these memory locations, at any given point in the computer program's execution, is called the program state. The executability of the personalised medicine treatment program 27 may depend on the execution environment in which it runs and the program state. The program state may be known as the program “heap”/state or the program Sigma, Σ. The program state can determine (and limit) what execution means. The program state of the personalised medicine treatment program 27 may be referred to as the treatment state. The execution engine 25 can construct a treatment state trace of the treatment state for each beat in the treatment over time throughout program execution. The execution engine 25 may output the treatment state trace to define a certificate of treatment as a form of in-use validation. In other words, the treatment state trace can provide evidence of treatment program execution that enforces safety of automated personalised medical treatments, and may provide an audit trail of outcomes over time over different treatment states.

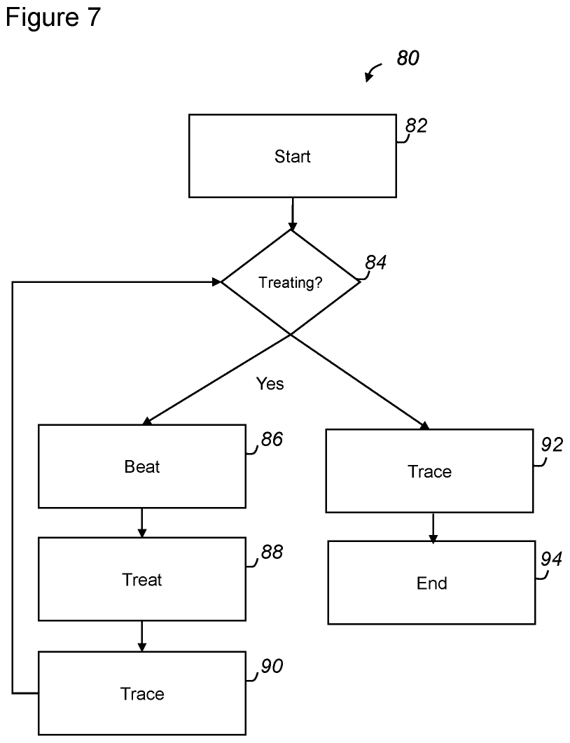

FIG. 7 illustrates a process of (or an algorithm for) executing a DSL personalised medicine treatment program according to an embodiment of the present disclosure. The execution engine 25 may perform the execution process of FIG. 7.

The process starts (step 82) and proceeds to a decision step 84 to determine if treatment is ongoing. If treatment is ongoing, the treatment program 27 proceeds through a beat step 86, a treatment step 88 and a trace step 90. Following the trace step 90, the treatment program 27 returns to decision step 84. The treatment program 27 may perform this loop at the frequency corresponding to the program beat. If at decision step 84 the program 27 determines that treatment is not ongoing (for example a treatment objective was met during the treat step 88 or a mandatory treatment ambition was breached), the program proceeds to a final trace step 92 before terminating at step 94.

The process comprises a “beat-treat-trace” loop, whilst the treatment is executing, with the final trace over treatment states as a certificate of treatment. The program beat step 86 may represent the smallest amount of consistent time within treatment execution. The beat step 86 may also determine when measurements should be made (e.g., measure blood pressure daily for hypertension treatment). Within the treatment package 20, various beat frequencies can be set. For example, the medical knowledge database 28 may stipulate microgram doses over 24 hrs, whereas the pathway program 30 may beat daily with milligram doses). Thus, the program beat may represent the shortest length of consistent time within treatment execution. The compiler 22 during compilation, or the execution engine 25 during runtime, may consolidate the various beat reference time units within the treatment package 20 to define the program beat. The compiler 22, or execution engine 25, may convert each beat reference time unit to a consistent program beat reference unit. Maintaining consistent time beat conversion can avoid a loss of precision. Consistency may be guaranteed when converting to both a faster time granularity (e.g., 1 day is 24 hours), and a slower time granularity (e.g., medical knowledge program 24 hours converted to pathway program daily beat). For each beat step 86, the execution engine 25 may determine what tasks (measurements, treatment titration criteria evaluation etc) to perform during the subsequent treat step 88.

During the program treat step 88, the execution engine can evaluate and can track all safety guardrails and terminating conditions (based on the treatment state/program state) to ensure patient safety. The execution engine may use formal methods to evaluate the safety guardrails and terminating conditions. If the execution engine 25 determines during the beat step 86 that treatment titration criteria should be evaluated, the execution engine 25 may also evaluate the titration criteria that modulate changes in treatment and output the change in the treatment to the patient, during the treat step 88. If the execution engine 25 determines during the beat step 86 that one or more measurements are required, the execution engine 25 may request the measurements from the patient during the treat step.

During the program trace step 90, 92 the execution engine outputs the “journaled” accumulation of treatment program states. As the program state changes over time, the accumulation over different program states can demonstrate that the safety guardrails and titration criteria have been satisfied throughout the duration of the treatment.

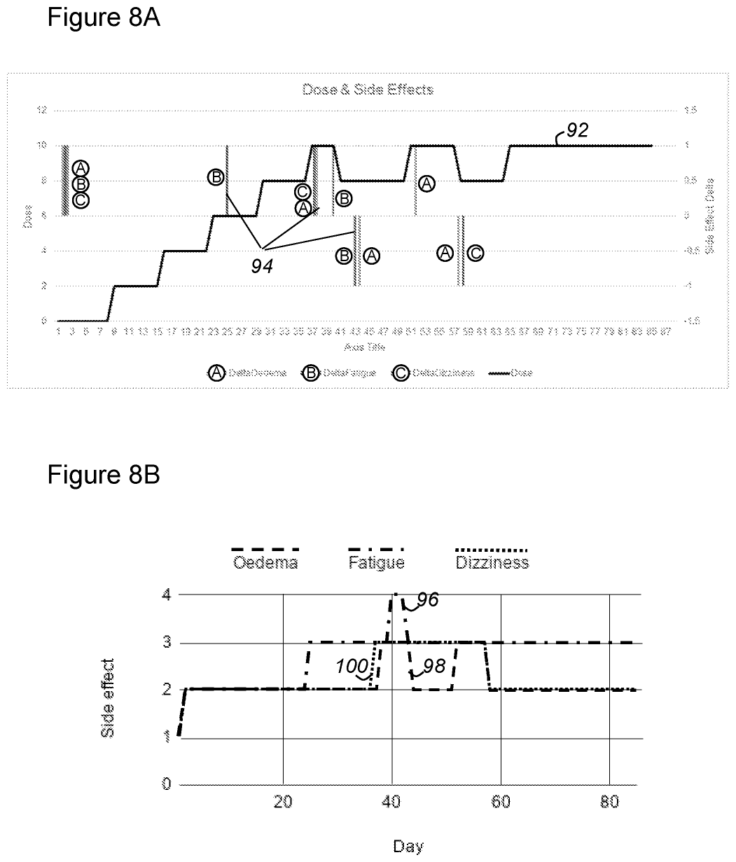

The treatment state trace may also enable visualisation of the treatment titration trajectory. FIGS. 8A and 8B illustrate such a visualisation for a personalised hypertension treatment and also illustrate how the program executes treatment titration criteria in response to changing side effects. FIG. 8A plots Amlodipine dosage 92 per day together with any daily changes in measured side effects. The left-hand vertical axis corresponds to Amlodipine dose in mg. The right-hand vertical axis corresponds to a measured change in side effect (an increasing side effect delta corresponding to an increase in side effect severity). Side effect changes are labelled for Oedema (A), Fatigue (B) and Dizziness (C). FIG. 8B plots the corresponding variation in raw values of side-effect levels for fatigue 96, oedema 98 and dizziness 100. The vertical axis in FIG. 8B corresponds to the side effect level, where 1=Baseline, 2=acceptable, 3=uncomfortable and 4=intolerable. The dosage and measured side effects are plotted for twelve weeks of daily measurements.

In an initial period from 0 to 35 days, the execution engine executes the treatment titration criteria and increases the dosage by 2 mg weekly (titration beat) because the side effects are all at an uncomfortable level or better (and other criteria indicate an increase is required (eg. Blood pressure measurements)). At days 35 to 39, a change in fatigue to an intolerable level coincides with approaching a maximum dosage (10 mg). The treatment program responds by decreasing the dosage because a patient intolerance has been detected via a satisfied treatment titration criteria. From day 43 onwards, the treatment program modulates the dosage between 8 and 10 mg in response to the variation in the patient's measured side effects. It will be appreciated that the side effect personalisation described above can enable different modulation profiles for an individual, for example such that an uncomfortable level of a side effect will result in a dosage reduction. In addition, the program may respond to adverse side effects by terminating the program due to a satisfied adverse side-effect treatment objective. The program 27 may output a treatment state trace with data of a similar type to that illustrates in FIGS. 8A and 8B. In this way, the treatment state trace can indicate the changes in side effect “level,” an improvement or deterioration in side effect tolerability and how these relate to Amlodipine dose changes.

In some examples, the execution engine 25 may be configured to operate the personalised medicine treatment program 27 in a plurality of treatment modes. The plurality of treatment modes may include an inform mode; a drive mode; and/or an automate mode. In the automate mode the treatment program 27 may direct the patient to change dose without clinician approval. In the drive mode, the treatment program 27 may provide a treatment change recommendation, such that a clinician can make the final decision. In the inform mode, the treatment program 27 may only output data such as measurement data or the treatment trace.

The execution engine 25 may operate the treatment program 27 in one of the plurality of modes based on a current treatment modality dosage (as defined by the initial dose or the most recent treatment modulation). For example, the execution engine may operate the treatment program:
in the automate mode if the current dosage is within a regulatory approved dosage range;
in the drive mode if the current dosage is outside the regulatory approved dosage range and within a drive dosage range; and/or
in the inform mode if the current dosage is outside both the regulatory approved dosage range and the drive dosage range.

The execution engine and the intermediate program form 26 may interact in a number of ways to define and execute the different modes of operation. For example, the execution engine may receive the regulatory approved dosage range (e.g. as defined in step 54 of FIG. 4) and/or the drive dosage range from the intermediate program form 26. Alternatively, the intermediate program form 26 may instead include a treatment ambition for each mode that includes a predicate evaluating the current dose against the regulatory approved dosage range and/or drive dosage range.

By operating in a plurality of different treatment modes, the treatment program 27 can provide an additional level of patient safety by only instructing and/or recommending dosage changes within a constrained safe dosage range. Such a mechanism can also reduce regulatory burden as regulators can easily recognise that such a breaker will prevent the treatment program providing unsafe dosage instructions or recommendation.

The validated DSL treatment package—the intermediate program form 26—may comprise an abstract syntax tree (AST). As some treatment information (measurements, patient inputs etc) is only available at run time, the AST may comprise a delayed computation mechanism for guaranteeing that the safety guardrails and titration modulation will remain true at every program state for all program states executed for the treatment program 27 (i.e., delayed computation is performed for every program state as treatment progresses).

The delayed execution structure may comprise various forms for the safety guardrails and treatment titration expressions as follows:
safety guardrails: each safety guardrail (explicit and implicit treatment objectives and treatment ambitions) may contain an expression that evaluates to true or false over the program state;
titration modulation: each titration expression may contain a series of delayed execution structures as:
   assumptions: an expression that evaluates to true/false over program state (i.e., all that must be true of Sigma before execution);
   ensures: an expression that evaluates to true/false over the program state before and after execution (i.e., all that must be the case after an update), with respect to the given initial conditions;
   implements: state to state expression over the original program state implementing the actual treatment modification.

The delayed-execution structures define mathematical logical predicates checked at run time by the execution engine 25 as the program executes with different program state values over time. The output for each check forms part of the treatment trace and the certification of treatment after program execution. This can provide an audit trail of evidence for how the treatment execution performed against its instructions as per the DSL programs.

### Safety Guardrail Definition

Following the explanation on execution and program state, we can return to the definition of safety guardrails (both implicit and explicit) in the DSL treatment package 20. The safety guardrails can be defined in the DSL as a set of safety treatment ambitions or as a set of treatment objectives. The treatment ambitions and the treatment objectives can be syntactically equivalent but should be treated differently during execution. The execution engine 25 can evaluate both treatment ambitions and objectives throughout program states over all treatment beats. Treatment objectives are terminating conditions: whenever they are satisfied (ie return true), the “beat-treat-trace” loop stops. Treatment ambitions are global treatment properties: they are to be kept satisfied (return true) throughout execution for all program states.

In some examples, the users can also define how the set of treatment objectives or treatment ambitions are checked as either “all” or “any”. This enables a strict (all) or loose (any) definition of safety for both ambitions and objectives. In some examples, by default, ambitions may be strict, and objectives may be loose. In some examples, variations on this default are possible: e.g., if objectives were strict (all), then only terminate the program when all objectives have been achieved.

In the DSL syntax, each of the safety guardrails may be defined by a named logical expression within a mandate, which can be defined as

name(mandate): expression;

Names should be unique to provide identity of what happened during treatment execution, as they participate in the treatment state tracing over time and in the certificate of treatment. The expression may comprise a formal method or predicate that can return only a true value or a false value.

Mandates can determine the scope of a safety guardrail. The mandate can be mandatory, desirable, or voluntary. Mandatory safety treatment ambitions relate to treatment properties that must be enforced (remain true) throughout treatment states during program execution. If a mandatory treatment ambition returns a false value, the treatment state trace will log an error and the treatment will terminate. As an example, a mandatory treatment ambition may define a toxic drug dosage that must never be exceeded. Mandatory safety treatment objectives relate to treatment conditions that will lead to the immediate termination of treatment (e.g., measurement of an adverse side effect).

Desirable or voluntary safety treatment ambitions relate to treatment properties that should be enforced throughout treatment states during execution. If a desirable treatment ambition returns a false value for a treatment state, a treatment fault occurs. The treatment program 27 can log the fault as part of the certificate of treatment. An example desirable treatment ambition may define a preference to avoid an uncomfortable side effect.

All treatment safety objectives are mandatory (no desirable treatment objectives), because voluntary termination of such objectives may lead to non-deterministic computation for which it is harder to ensure safety.

The different mandates can categorise the safety guardrails according to severity level. In this way, the safety guardrails can be differentiated between failures such as a toxic drug dosage, and faults or a local error within a module or its parts, such as a side effect personalisation issue. Safety guardrails with desirable mandates allow local errors to occur without termination of the treatment. Instead, the personalised medicine treatment program can implement mitigation measures to restore the safety guardrail treatment ambition to a true value for a future treatment state. For example, treatment modulation expressions may modulate a dosage level dependent when the treatment ambition returns a false value, to reduce a side effect severity level and restore patient comfort. The mandate strategy can enable a failure and fault management procedure: mandates can assign a perceived impact and mitigation mechanism for possible faults if they remain/occur during program execution. In this way, treatment safety is enforced through program state tracing as evidence for the certification of treatment safety.

1. A method of producing an intermediate program representation for an executable personalised medicine treatment program, comprising:
providing a domain specific language environment comprising a personalised-medicine programming language syntax and a user interface;
selecting a medical knowledge database for a medical condition;
receiving user input to build, using the personalised-medicine programming language syntax and the medical knowledge database, a treatment package for implementing one or more treatment modalities for the medical condition, the treatment package including:
treatment objectives defining terminating conditions of the one or more treatment modalities;
safety guardrails for ensuring patient safety; and
treatment titration definitions for modulating the one or more treatment modalities; and
validating the treatment package to demonstrate that the treatment objectives, safety guardrails and treatment titration definitions will be satisfied during execution of the personalised medicine treatment program.

2. (canceled)

3. The method of claim 1, wherein validating the treatment package comprises performing well-formedness checks against semantic criteria using formal methods.

4. The method of claim 1, wherein building the treatment package comprises defining the treatment objectives, safety guardrails and/or treatment titration definitions using formal methods.

5. The method of claim 1, wherein the treatment objectives, safety guardrails and/or treatment titration definitions comprise a logical construct for evaluation during execution of the personalised medicine treatment program, wherein the logical construct is mathematically formalised.

6. The method of claim 5, wherein the logical construct is suitable for evaluation during execution using one or more formal methods.

7. (canceled)

8. The method of claim 1, further comprising receiving user input to define one or more of:
patient measurements for the one or more treatment modalities;
dosages for the one or more treatment modalities;
explicit treatment objectives;
explicit treatment ambitions defining one or more invariant properties for satisfying throughout the treatment for the one or more treatment modalities; and
explicit treatment titration definitions.

9. The method of claim 1, wherein the safety guardrails comprise explicit safety guardrails defined by one or more explicit treatment objectives and/or one or more explicit treatment ambitions.

10. The method of claim 1, further comprising compiling the treatment package, wherein compiling the treatment package comprises one or more of:
expanding implicitly defined syntactic and semantic constructs within the user defined treatment package:
inferring and overriding information from linkages within the treatment package; and
defining implicit safety guardrails in the treatment package.

11-12. (canceled)

13. The method of claim 10, wherein the implicit safety guardrails include one or more of:
an implicit adverse side effect treatment objective requiring immediate termination of the treatment if an adverse side effect occurs during execution of the personalised medicine treatment program;
an implicit toxicity treatment ambition requiring a modality dosage to remain within a safe toxicity range, as defined by the medical knowledge database, throughout execution of the personalised medicine treatment program;
an implicit sensor treatment ambition requiring sensor measurements to remain within a plausible measurement range throughout execution of the personalised medicine treatment program; and
an implicit side effect combination treatment objective requiring termination of the treatment if a combination of measured side effects indicative of an overdose is determined during execution of the personalised medicine treatment program.

14. The method of claim 10, wherein:
receiving user input comprises receiving user input to define one or more personal side effect tolerances; and
the implicit safety guardrails include a desirable side effect treatment ambition derived from personal side effect tolerances.

15. The method of claim 1, further comprising receiving user input to personalise the treatment package.

16. The method of claim 15, wherein receiving user input to personalise the treatment package comprises one or more of:
receiving user input to personalise one or more of the treatment titration definitions;
receiving user input to personalise one or more initial treatment conditions; and
receiving user input to define one or more personal side effect tolerances.

17-20. (canceled)

21. The method of claim 1, wherein the treatment objectives, safety guardrails and/or treatment titration definitions comprise a logical predicate, wherein the logical predicate comprises a dependence on a program state of the personalised medicine treatment program.

22. (canceled)

23. The method of claim 1, wherein the treatment titration definitions comprise one or more treatment titration criteria for evaluation during execution of the personalised medicine treatment program.

24. The method of claim 23, wherein the treatment titration criteria comprise instructions for modulating the treatment comprising modulating any of:
a measurement frequency;
a dose frequency; and/or
a dose amount.

25. The method of claim 23, wherein the treatment titration criteria have a dependency on:
one or more side effect measurements; and/or
one or more physiological measurements.

26. The method of claim 1, wherein the treatment titration definitions specify a titration beat frequency for evaluating treatment modulation definitions during execution of the personalised medicine treatment program.

27. The method of claim 1, wherein the intermediate program form is configured to indicate a compliance with one or more regulatory approved dosage ranges during execution of the personalised medicine treatment program to enable operation of the personalised medicine treatment program in one of a plurality of modes.

28. An intermediate program representation for an executable personalised medicine treatment program, comprising:
a treatment package for implementing one or more treatment modalities for a medical indication, the treatment package including:
treatment objectives defining terminating conditions of the one or more treatment modalities;
safety guardrails for ensuring patient safety; and
treatment titration definitions for modulating the one or more treatment modalities,
wherein the treatment package is built using a medical knowledge database and a personalised-medicine programming language syntax of a domain specific language environment.

29-40. (canceled)

41. A personalised medicine treatment program comprising the intermediate program form of claim 28.

42-54. (canceled)
